(19) United States
(12) Patent Application Publication
(10) Pub. No.: US 2012/0015968 A1
(43) Pub. Date: Jan. 19, 2012

(54) USE OF PYRIMIDYLAMINOBENZAMIDE DERIVATIVES FOR THE TREATMENT OF DISORDERS MEDIATED BY THE LEUCINE ZIPPER-AND STERILE ALPHA MOTIF-CONTAINING KINASE (ZAK)

(75) Inventors: Doriano Fabbro, Arlesheim (CH); Paul W. Manley, Arlesheim (CH)

(73) Assignee: NOVARTIS AG, Basel (CH)

(21) Appl. No.: 13/254,261

(22) PCT Filed: Mar. 5, 2010
(86) PCT No.: PCT/EP10/52797

§ 371 Date: Oct. 5, 2011

(30) Foreign Application Priority Data
Mar. 6, 2009 (EP) 09154507.9
Jul. 27, 2009 (EP) 09166437.5

(51) Int. Cl. A61K 31/506; A61P 9/00; A61P 35/00

(52) U.S. Cl. 514/275

(57) ABSTRACT

The invention relates to the use of a pyrimidylaminobenzamide derivative of formula I

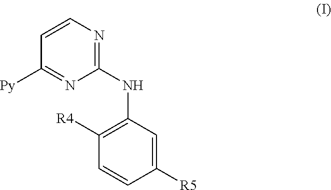

wherein the radicals have the meanings as defined herein, or of a pharmaceutically acceptable salt thereof for the manufacture of pharmaceutical compositions for use in the treatment of disorders mediated by ZAK, to the use of a pyrimidylaminobenzamide derivatives of formula I or pharmaceutically acceptable salt thereof in the treatment of disorders mediated by ZAK, and to a method of treating warm-blooded animals including humans suffering from disorders mediated by ZAK by administering to said warm-blooded animal in need of such treatment an effective dose of a pyrimidylaminobenzamide of formula I or a pharmaceutically acceptable salt thereof.

The invention relates to the use of a pyrimidylaminobenzamide derivatives of formula I as defined below or a pharmaceutically acceptable salt thereof for the manufacture of pharmaceutical compositions for use in the treatment of disorders mediated by ZAK, to the use of a pyrimidylaminobenzamide derivative of formula I or a pharmaceutically acceptable salt thereof in the treatment of disorders mediated by ZAK, and to a method of treating warm-blooded animals including humans suffering from disorders mediated by ZAK by administering to a said animal in need of such treatment an effective dose of a pyrimidylaminobenzamide derivative of formula I or a pharmaceutically acceptable salt thereof.

Leucine zipper- and sterile alpha motif-containing kinase (ZAK) is a serine-threonine kinase that belongs to the MAPKKK family of signal transduction molecules (Gross, E. A., at al, J. Biol. Chem., 277; 13873-13882, 2002).

By searching for sequences similar to yeast sterile-20 (Ste20), followed by screening a placenta cDNA library and 5-prime RACE, Liu at al cloned ZAK (Liu, T.-C., et al; Biochem. Biophys. Res. Commun. 274: 811-816, 2000). The deduced 800-amino acid protein has a calculated molecular mass of 91 kD. ZAK contains an N-terminal kinase catalytic domain, followed by a leucine zipper motif and a sterile-alpha motif (SAM). Northern blot analysis detected highest expression of a 3.0-kb ZAK transcript in heart, placenta, lung, liver, and pancreas.

Gotoh, I., at al. (J. Biol. Chem., 276: 4276-4286, 2001.) cloned 2 alternatively spliced forms of mouse Zak, which they designated Mltk-alpha and Mltk-beta. The deduced 803- and 454-amino acid proteins have calculated molecular masses of 91.7 and 51.3 kD, respectively. Both proteins contain an N-terminal kinase domain followed by a leucine-zipper motif, and both have a nuclear export signal. The 2 proteins differ in their C-terminal sequences, with Mltk-alpha having a SAM domain, and Mltk-beta having a sequence similar to the C-terminal region of TAK1 (MAP3K7). Northern blot analysis of human tissues detected a transcript of about 7.7 kb expressed at highest levels in heart and skeletal muscle. Minor transcripts of about 3.3 and 1.6 kb were also detected in heart and skeletal muscle. Upon transfection in COS-7 cells, mouse Mltk-alpha and Mltk-beta localized to the cytoplasm. Inhibition of nuclear export increased the nuclear accumulation of both proteins.

By screening a human Jurkat T-cell cDNA expression library for MAPK cascade members, followed by 5-prime RACE, Gross, E. A. et al (loc. cit.) identified 2 ZAK splice variants, MRK-alpha and MRK-beta, which differ at their 3-prime ends. The deduced 800- and 456-amino acid proteins have calculated molecular masses of 91.1 and 51.5 kD, respectively. Human MRK-alpha and MRK-beta have the same protein domain structure as mouse Mk-alpha and Mltk-beta. The common kinase domain of the human MRK isoforms shares 52% similarity with those of MLK1 (MAP3K9) and MLK2 (MAP3K10), and it shares 47% identity with that of TAK1. Northern blot analysis detected a prominent transcript of 7.5 kb and less abundant transcripts of 3.8 and 1.6 kb in all tissues examined. Highest expression was detected in skeletal muscle and head, and weak expression was detected in brain and kidney. Transcript-specific probes identified the 3.8-kb transcript as MRK-alpha and the 7.5-kb transcript as MRK-beta.

By immunoprecipitation of a transfected human hepatoma cell line, Liu et al (loc. cit.) determined that ZAK can form oligomers. By in vitro kinase assays, they found that ZAK activated JNK/SAPK1 (MAPL8) and NFKB. Overexpression of ZAK resulted in apoptosis.

Using cotransfection assays in COS-7 cells, Gotoh et al (loc. cit.) found that both mouse Mltk-alpha and Mltk-beta activated Erk2 (MAPK1), Jnk, p38 (MAPK14), and Erk5 (MAPK7). Both Mltks activated all MAP kinase pathways tested by phosphorylating and activating the respective MAP kinase kinases. Mltk-alpha and Mltk-beta were also activated, by auto-phosphorylation in response to osmotic shock with hyperosmolar media. Expression of Mltk-alpha, but not Mltk-beta, in mouse fibroblasts resulted in disruption of actin stress fibers and dramatic morphologic changes. A kinase-dead mutant of Mltk-alpha did not cause these changes, and inhibition of the p38 pathway significantly blocked Mltk-alpha-induced stress fiber disruption and morphologic changes.

Gross et al (loc. cit.) found that MRK-beta expressed in transfected COS-1 cells exhibited autophosphorylation and kinase activity toward a generic test substrate. Mutation of a critical lysine (lys45) to alanine abolished these activities. Using a combination of solid-phase protein kinase assays, transient transfections, and analysis of the effect of transfected human MRK-beta on endogenous proteins in transfected canine kidney cells, Gross et al (loc. cit.) found that MRK-beta preferentially activated ERK5/p38-gamma via MKK3 (MAP2K3)/MKK6 (MAP2K6) and JNK via MKK4 (MAP2K4)/MKK7 (MAP2K7). Expression of MRK increased the population of cells in the G2/M phase of the cell cycle, whereas dominant-negative MRK attenuated the G2 arrest caused by gamma radiation. In addition, exposure of cells to gamma radiation induced MRK activity. Gross et al (loc. cit.) concluded that MRK may mediate gamma radiation signaling leading to cell cycle arrest and that MRK activity is necessary for cell cycle checkpoint regulation in cells.

Yang found that mammalian ZAK activated Jnk through Mkk7. Expression of kinase-dead Zak in mouse fibroblasts disrupted actin stress fibers and caused morphologic changes (Yang, J.-J., Biochem. Biophys. Res. Commun. 297: 105-110, 2002). Expression of wildtype Zak increased the number of cells in the G2/M phase of the cell cycle. Yang concluded that ZAK activity may be involved in regulating actin organization and in G2 arrest.

By immunoprecipitation of cotransfected human embryonic kidney cells, Yang found direct interaction between epitope-tagged ZZAPK (ZNF33A) and ZAK (Yang, J.-J., Biochem. Biophys. Res. Commun. 301: 71-77, 2003). Mutation analysis indicated that the SAM domain of ZAK was required to bind ZZAPK. By coexpression in a rat fibroblast cell line. Yang found that ZZAPK countered the effect of ZAK on G2/M cell cycle arrest.

Zak is a positive mediator of cell hypertrophy in cultured rat cardiac myocytes. Huang et al. found that expression of a dominant-negative Zak protein inhibited features characteristic of TGF-beta-induced cardiac hypertrophy in these cultures, including increased cell size, elevated expression of atrial natriuretic factor (ANF), and increased organization of actin fibers (Huang, C.-Y. et al., Biochem. Biophys. Res. Commun. 324: 424-431, 2004). Furthermore, dominant-negative Mkk7 blocked both Tgf-beta and Zak-induced Anf expression. Huang concluded that ZAK mediates TGF-beta-induced cardiac hypertrophy via a TGF-beta-ZAK-MKK7-ANF signaling pathway.

ZAK over-expression is associated with cardiac hypertrophy (Huang, et al. BBRC 2004; 324:973)

ZAK signaling was found to induce MMP-2 activity and at the same time to reduce MMP-9 activity. Taken together, ZAK activity may be a suitable intervention to prevent cardiac fibrosis progression.

As result of E. coli infection, shiga toxicity causes hemolytic uremic syndrome. The toxicity of the Shiga toxin with the involvement of kinase activation (Korcheva, et al. Am J Pathol 2005; 166:323) appears to be controlled by activation of the ZAK kinase (Jandhyala, et al. Cellular Microbiology 2008; 10:1468).

It was now surprisingly found that the pyrimidylaminobenzamide derivatives of formula can be used for the treatment of disorders mediated by ZAK in view of the observation that ZAK is a target of the pyrimidylaminobenzamide derivatives of formula I.

Hence, the present invention relates to the use of pyrimidylaminobenzamide derivatives of formula I

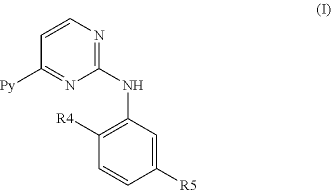

wherein

(a) Py denotes 3-pyridyl,

R₅ denotes —C(O)—NR₁R₂,

R₁ represents hydrogen, lower alkyl, lower alkoxy-lower alkyl, acyloxy-lower alkyl, carboxy-lower alkyl, lower alkoxycarbonyl-lower alkyl, or phenyl-lower alkyl;

R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising zero, one two or three ring nitrogen atoms and zero or one oxygen atom and zero or one sulfur atom, which groups in each case are unsubstituted or mono- or polysubstituted; and

R₃ represents hydroxy, lower alkoxy, acyloxy, carboxy, lower alkoxycarbonyl, carbamoyl, N-mono- or N,N-disubstituted carbamoyl, amino, mono- or disubstituted amino, cycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted;

or wherein R₁ and R₂ together represent alkylene with four, five or six carbon atoms optionally mono- or disubstituted by lower alkyl, cycloalkyl, heterocyclyl, phenyl, hydroxy, lower alkoxy, amino, mono- or disubstituted amino, oxo, pyridyl, pyrazinyl or pyrimidinyl; benzalkylene with four or five carbon atoms; oxaalkylene with one oxygen and three or four carbon atoms; or azaalkylene with one nitrogen and three or four carbon atoms wherein nitrogen is unsubstituted or substituted by lower alkyl, phenyl-lower alkyl, lower alkoxycarbonyl-lower alkyl, carboxy-lower alkyl, carbamoyl-lower alkyl, N-mono- or N,N-disubstituted carbamoyl-lower alkyl, cycloalkyl, lower alkoxycarbonyl, carboxy, phenyl, substituted phenyl, pyridinyl, pyrimidinyl, or pyrazinyl;

R₄ represents hydrogen, lower alkyl, or halogen; or

(b) Py denotes 5-pyrimidyl, R₅ denotes —N(R₁)—C(O)—R₂,

R₁ is hydrogen, R₂ is [[(3S)-3-(dimethylamino)-1-pyrrolidinyl]-methyl]-3-(trifluoromethyl)phenyl and R₄ is methyl;

or of a pharmaceutically acceptable salt thereof alone or in combination with other active compounds for the preparation of a pharmaceutical composition for the treatment of disorders mediated by ZAK.

The expression “disorders mediated by ZAK” as used herein include, but are not limited to, hemolytic uremic syndrome, cardiac hypertrophy, cardiac fibrosis progression, and ovarian cancer, especially ovarian cancer harboring at least one ZAK mutation.

The terms “treatment” or “therapy” refer to the prophylactic or preferably therapeutic (including but not limited to palliative, curing, symptom-alleviating, symptom-reducing, kinase-regulating and/or kinase-inhibiting) treatment of the diseases disclosed herein.

In one embodiment of the present invention, preference is given to pyrimidylamino-benzamide derivatives of formula I, wherein py is 3-pyridyl, R₅ denotes —C(O)—NR₁R₂, and wherein the radicals mutually independently of each other have the following meanings:

R₁ represents hydrogen, lower alkyl, lower alkoxy-lower alkyl, acyloxy-lower alkyl, carboxy-lower alkyl, lower alkoxycarbonyl-lower alkyl, or phenyl-lower alkyl; more preferably hydrogen;

R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted;

R₃ represents hydroxy, lower alkoxy, acyloxy, carboxy, lower alkoxycarbonyl, carbamoyl, N-mono- or N,N-disubstituted carbamoyl, amino, mono- or disubstituted amino, cycloalkyl, heterocyclyl, an aryl group, or a mono or bicyclic heteroaryl group comprising zero, one two or three ring nitrogen atoms and zero or one oxygen atom and zero or one sulfur atom, which groups in each case are unsubstituted or mono- or polysubstituted; and

R₄ represents lower alkyl, especially methyl.

A preferred pyrimidylaminobenzamide derivative is 4-methyl-3-[[4-(3-pyridinyl)-2-pyrimidinyl]amino]-N-[5-(4-methyl-1H-imidazol-1-yl)-3-(trifluoromethyl)phenyl]benzamide, also known as “nilotinib”.

The general terms used hereinbefore and hereinafter preferably have within the context of this disclosure the following meanings, unless otherwise indicated:

The prefix “lower” denotes a radical having up to and including a maximum of 7, especially up to and including a maximum of 4 carbon atoms, the radicals in question being either linear or branched with single or multiple branching.

Where the plural form is used for compounds, salts, and the like, this is taken to mean also a single compound, salt, or the like.

Lower alkyl is preferably alkyl with from and including 1 up to and including 7, preferably from and including 1 to and including 4, and is linear or branched; preferably, lower alkyl is butyl, such as n-butyl, sec-butyl, isobutyl, tert-butyl, propyl, such as n-propyl or isopropyl, ethyl or methyl. Preferably lower alkyl is methyl, propyl or tert-butyl.

Lower acyl is preferably formyl or lower alkylcarbonyl, in particular acetyl.

An aryl group is an aromatic radical which is bound to the molecule via a bond located at an aromatic ring carbon atom of the radical. In a preferred embodiment, aryl is an aromatic radical having 6 to 14 carbon atoms, especially phenyl, naphthyl, tetrahydronaphthyl, fluorenyl or phenanthrenyl, and is unsubstituted or substituted by one or more, preferably up to three, especially one or two substituents, especially selected from amino, mono- or disubstituted amino, halogen, lower alkyl, substituted lower alkyl, lower alkenyl, lower alkynyl, phenyl, hydroxy, etherified or esterified hydroxy, nitro, cyano, carboxy, esterified carboxy, alkanoyl, benzoyl, carbamoyl, N-mono- or N,N-disubstituted carbamoyl, amidino, guanidino, ureido, mercapto, sulfo, lower alkylthio, phenylthio, phenyl-lower alkylthio, lower alkylphenylthio, lower alkylsulfinyl, phenylsulfinyl, phenyl-lower alkylsulfinyl, lower alkylphenylsulfinyl, lower alkylsulfonyl, phenylsulfonyl, phenyl-lower alkylsulfonyl, lower alkylphenylsulfonyl, halogen-lower alkylmercapto, halogen-lower alkylsulfonyl, such as especially trifluoromethanesulfonyl, dihyciroxybora (—B(OH)₂), heterocyclyl, a mono- or bicyclic heteroaryl group and lower alkylene dioxy bound at adjacent C-atoms of the ring, such as methylene dioxy. Aryl is more preferably phenyl, naphthyl or tetrahydronaphthyl, which in each case is either unsubstituted or independently substituted by one or two substituents selected from the group comprising halogen, especially fluorine, chlorine, or bromine; hydroxy; hydroxy etherified by lower alkyl, e.g. by methyl, by halogen-lower alkyl, e.g. trifluoromethyl, or by phenyl; lower alkylene dioxy bound to two adjacent C-atoms, e.g. methylenedioxy, lower alkyl, e.g. methyl or propyl; halogen-lower alkyl, e.g. trifluoromethyl; hydroxy-lower alkyl, e.g. hydroxymethyl or 2-hydroxy-2-propyl; lower alkoxy-lower alkyl; e.g. methoxymethyl or 2-methoxyethyl; lower alkoxycarbonyl-lower alkyl, e.g. methoxy-carbonylmethyl; lower alkynyl, such as 1-propynyl; esterified carboxy, especially lower alkoxycarbonyl, e.g. methoxycarbonyl, n-propoxy carbonyl or iso-propoxy carbonyl; N-mono-substituted carbamoyl, in particular carbamoyl monosubstituted by lower alkyl, e.g. methyl, n-propyl or iso-propyl; amino; lower alkylamino, e.g. methylamino; di-lower alkylamino, e.g. dimethylamino or diethylamino; lower alkylene-amino, e.g. pyrrolidino or piperidino; lower oxaalkylene-amino, e.g. morpholino, lower azaalkylene-amino, e.g. piperazino, acylamino, acetylamino or benzoylamino; lower alkylsulfonyl, e.g. methylsulfonyl; sulfamoyl; or phenylsulfonyl.

A cycloalkyl group is preferably cyclopropyl, cyclopentyl, cyclohexyl or cycloheptyl, and may be unsubstituted or substituted by one or more, especially one or two, substitutents selected from the group defined above as substitutents for aryl, most preferably by lower alkyl, such as methyl, lower alkoxy, such as methoxy or ethoxy, or hydroxy, and further by oxo or fused to a benzo ring, such as in benzcyclopentyl or benzcyclohexyl.

Substituted alkyl is alkyl as last defined, especially lower alkyl, preferably methyl; where one or more, especially up to three, substituents may be present, primarily from the group selected from halogen, especially fluorine, amino, N-lower alkylamino, N,N-di-lower alkylamino, N-lower alkanoylamino, hydroxy, cyano, carboxy, lower alkoxycarbonyl, and phenyl-lower alkoxycarbonyl. Trifluoromethyl is especially preferred.

Mono- or disubstituted amino is especially amino substituted by one or two radicals selected independently of one another from lower alkyl, such as methyl; hydroxy-lower alkyl, such as 2-hydroxyethyl; lower alkoxy lower alkyl, such as methoxy ethyl; phenyl-lower alkyl, such as benzyl or 2-phenylethyl; lower alkanoyl, such as acetyl; benzoyl; substituted benzoyl, wherein the phenyl radical is especially substituted by one or more, preferably one or two, substituents selected from nitro, amino, halogen. N-lower alkylamino, N,N-di-lower alkylamino, hydroxy, cyano, carboxy, lower alkoxycarbonyl, lower alkanoyl, and carbamoyl; and phenyl-lower alkoxycarbonyl, wherein the phenyl radical is unsubstituted or especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl, and carbamoyl; and is preferably N-lower alkylamino, such as N-methylamino, hydroxy-lower alkylamino, such as 2-hydroxyethylamino or 2-hydroxypropyl, lower alkoxy lower alkyl, such as methoxy ethyl, phenyl-lower alkylamino, such as benzylamino, N,N-di-lower alkylamino, N-phenyl-lower alkyl-N-lower alkylamino, N,N-di-lower alkylphenylamino, lower alkanoylamino, such as acetylamino, or a substituent selected from the group comprising benzoylamino and phenyl-lower alkoxycarbonylamino, wherein the phenyl radical in each case is unsubstituted or especially substituted by nitro or amino, or also by halogen, amino, N-lower alkylamino, N,N-di-lower alkylamino, hydroxy, cyano, carboxy, lower alkoxycarbonyl, lower alkanoyl, carbamoyl or aminocarbonylamino. Disubstituted amino is also lower alkylene-amino, e.g. pyrrolidino, 2-oxopyrrolidino or piperidino; lower oxaalkylene-amino, e.g. morpholino, or lower azaalkylene-amino, e.g. piperazino or N-substituted piperazino, such as N-methylpiperazino or N-methoxycarbonylpiperazino.

Halogen is especially fluorine, chlorine, bromine, or iodine, especially fluorine, chlorine, or bromine.

Etherified hydroxy is especially C₈-C₂₀alkyloxy, such as n-decyloxy, lower alkoxy (preferred), such as methoxy, ethoxy, isopropyloxy, or tert-butyloxy, phenyl-lower alkoxy, such as benzyloxy, phenyloxy, halogen-lower alkoxy, such as trifluoromethoxy, 2,2,2-trifluoroethoxy or 1,1,2,2-tetrafluoroethoxy, or lower alkoxy which is substituted by mono- or bicyclic hetero-aryl comprising one or two nitrogen atoms, preferably lower alkoxy which is substituted by imidazolyl, such as 1H-imidazol-1-yl, pyrrolyl, benzimidazolyl, such as 1-benzimidazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, indolyl or thiazolyl.

Esterified hydroxy is especially lower alkanoyloxy, benzoyloxy, lower alkoxycarbonyloxy, such as tert-butoxycarbonyloxy, or phenyl-lower alkoxycarbonyloxy, such as benzyloxycarbonyloxy.

Esterified carboxy is especially lower alkoxycarbonyl, such as tert-butoxycarbonyl, iso-propoxycarbonyl, methoxycarbonyl or ethoxycarbonyl, phenyl-lower alkoxycarbonyl or phenyloxycarbonyl.

Alkanoyl is primarily alkylcarbonyl, especially lower alkanoyl, e.g. acetyl.

N-Mono- or N,N-disubstituted carbamoyl is especially substituted by one or two substituents independently selected from lower alkyl, phenyl-lower alkyl and hydroxy-lower alkyl, or lower alkylene, oxa-lower alkylene or aza-lower alkylene optionally substituted at the terminal nitrogen atom.

A mono- or bicyclic heteroaryl group comprising zero, one, two or three ring nitrogen atoms and zero or one oxygen atom and zero or one sulfur atom, which groups in each case are unsubstituted or mono- or polysubstituted, refers to a heterocyclic moiety that is unsaturated in the ring binding the heteroaryl radical to the rest of the molecule in formula I and is preferably a ring, where in the binding ring, but optionally also in any annealed ring, at least one carbon atom is replaced by a heteroatom selected from the group consisting of nitrogen, oxygen and sulfur; where the binding ring preferably has 5 to 12, more preferably 5 or 6 ring atoms; and which may be unsubstituted or substituted by one or more, especially one or two, substitutents selected from the group defined above as substitutents for aryl, most preferably by lower alkyl, such as methyl, lower alkoxy, such as methoxy or ethoxy, or hydroxy. Preferably the mono- or bicyclic heteroaryl group is selected from 2H-pyrrolyl, pyrrolyl, imidazolyl, benzimidazolyl, pyrazolyl, indazolyl, purinyl, pyridyl, pyrazinyl, pyrimidinyl, pyridazinyl, 4H-quinolizinyl, isoquinolyl, quinolyl, phthalazinyl, naphthyridinyl, quinoxalyl, quinazolinyl, quinolinyl, pteridinyl, indolizinyl, 3H-indolyl, indolyl, isoindolyl, oxazolyl, isoxazolyl, thiazolyl, isothiazolyl, triazolyl, tetrazolyl, furazanyl, benzo[d]pyrazolyl, thienyl and furanyl. More preferably the mono- or bicyclic heteroaryl group is selected from the group consisting of pyrrolyl, imidazolyl, such as 1H-imidazol-1-yl, benzimidazolyl, such as 1-benzimidazolyl, indazolyl, especially 5-indazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, especially 4- or 8-quinolinyl, indolyl, especially 3-indolyl, thiazolyl, benzo[d]pyrazolyl, thienyl, and furanyl. In one preferred embodiment of the invention the pyridyl radical is substituted by hydroxy in ortho position to the nitrogen atom and hence exists at least partially in the form of the corresponding tautomer which is pyridin-(1H)2-one. In another preferred embodiment, the pyrimidinyl radical is substituted by hydroxy both in position 2 and 4 and hence exists in several tautomeric forms, e.g. as pyrimidine-(1H, 3H)2,4-dione.

Heterocyclyl is especially a five, six or seven-membered heterocyclic system with one or two heteroatoms selected from the group comprising nitrogen, oxygen, and sulfur, which may be unsaturated or wholly or partly saturated, and is unsubstituted or substituted especially by lower alkyl, such as methyl, phenyl-lower alkyl, such as benzyl, oxo, or heteroaryl, such as 2-piperazinyl; heterocyclyl is especially 2- or 3-pyrrolidinyl, 2-oxo-5-pyrrolidinyl, piperidinyl, N-benzyl-4-piperidinyl, N-lower alkyl-4-piperidinyl, N-lower alkyl-piperazinyl, morpholinyl, e.g. 2- or 3-morpholinyl, 2-oxo-1H-azepin-3-yl, 2-tetrahydrofuranyl, or 2-methyl-1,3-dioxolan-2-yl.

Pyrimidylaminobenzamide derivatives within the scope of formula I, wherein py is 3-pyridyl, and R₅ denotes —C(O)—NR₁R₂, and the process for their manufacture are disclosed in WO 04/005281 published on Jan. 15, 2004 which is hereby incorporated into the present application by reference. The inhibition of ZAK activity by INNO-406 was also reported by U. Rix at al, Leukemia (2010) 24, 44-50. Using biotinylated myelin basic protein as a substrate, INNO-406 inhibited ZAK activity with an IC50 of 73 nM (U. Rix, loc. cit., p. 48).

The pyrimidylaminobenzamide of formula I wherein (b) Py denotes 5-pyrimidyl, R₅ denotes —N(R₁)—C(O)—R₂, R₁ is hydrogen, R₂ is [[(3S)-3-(dimethylamino)-1-pyrrolidinyl]methyl]-3-(trifluoromethyl)phenyl and R₄ is methyl; is also known as INNO-406. The compound, its manufacture and pharmaceutical compositions suitable for its administration are disclosed in EPI 533304A.

Pharmaceutically acceptable salts of pyrimidylaminobenzamide derivatives of formula I, wherein py is 3-pyridyl and R₅ denotes —C(O)—NR₁R₂, are especially those disclosed in WO2007/015871. In one preferred embodiment nilotinib is employed in the form of its hydrochloride monohydrate. WO2007/015870 discloses certain polymorphs of nilotinib and pharmaceutically acceptable salts thereof useful for the present invention.

The pyrimidylaminobenzamide derivatives of formula I, wherein py is 3-pyridyl and R₅ denotes —C(O)—NR₁R₂, can be administered by any route including orally, parenterally, e.g., intraperitoneally, intravenously, intramuscularly, subcutaneously, intratumorally, or rectally, or enterally. Preferably, the pyrimidylaminobenzamide derivatives of formula I, wherein py is 3-pyridyl and R₅ denotes —C(O)—NR₁R₂, is administered orally, preferably at a daily dosage of 50-2000 mg. A preferred oral daily dosage of nilotinib is 200-1200 mg, e.g. 800 mg, administered as a single dose or divided into multiple doses, such as twice daily dosing.

INNO-406 can be administered orally twice daily in a dose of 200 to 300 mg, e.g. 240 mg.

Usually, a small dose is administered initially and the dosage is gradually increased until the optimal dosage for the host under treatment is determined. The upper limit of dosage is that imposed by side effects and can be determined by trial for the host being treated.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

The person skilled in the pertinent art is fully enabled to select a relevant test model to prove the hereinbefore and hereinafter indicated therapeutic indications and beneficial effects. The pharmacological activity is, for example, demonstrated in well established in vitro and in vivo test procedures, or in a clinical study as essentially described hereinafter.

1-4. (canceled)

5. A method of treating or preventing disorders mediated by ZAK comprising administering a pyrimidylaminobenzamide derivative of formula (I): 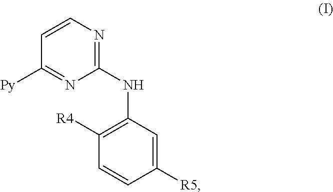
wherein
(a) Py denotes 3-pyridyl,
R₅ denotes —C(O)—NR₁R₂,
R₁ represents hydrogen, lower alkyl, lower alkoxy-lower alkyl, acyloxy-lower alkyl, carboxy-lower alkyl, lower alkoxycarbonyl-lower alkyl, or phenyl-lower alkyl;
R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising 0-, 1-, 2- or 3-ring nitrogen atoms and 0 or 1 oxygen atom and 0 or 1 sulfur atom, which groups in each case are unsubstituted or mono- or poly-substituted; and
R₃ represents hydroxy, lower alkoxy, acyloxy, carboxy, lower alkoxycarbonyl, carbamoyl, N-mono- or N,N-di-substituted carbamoyl, amino, mono- or di-substituted amino, cycloalkyl, heterocyclyl, an aryl group, or a mono- or bi-cyclic heteroaryl group comprising 0-, 1-, 2- or 3-ring nitrogen atoms and 0 or 1 oxygen atom and 0 or 1 sulfur atom, which groups in each case are unsubstituted or mono- or poly-substituted; or
R₁ and R₂, together, represent alkylene with 4, 5 or 6 carbon atoms optionally mono- or di-substituted by lower alkyl, cycloalkyl, heterocyclyl, phenyl, hydroxy, lower alkoxy, amino, mono- or di-substituted amino, oxo, pyridyl, pyrazinyl or pyrimidinyl; benzalkylene with 4 or 5 carbon atoms; oxaalkylene with 1 oxygen and 3 or 4 carbon atoms; or azaalkylene with 1 nitrogen and 3 or 4 carbon atoms, wherein nitrogen is unsubstituted or substituted by lower alkyl, phenyl-lower alkyl, lower alkoxycarbonyl-lower alkyl, carboxy-lower alkyl, carbamoyl-lower alkyl, N-mono- or N,N-di-substituted carbamoyl-lower alkyl, cycloalkyl, lower alkoxycarbonyl, carboxy, phenyl, substituted phenyl, pyridinyl, pyrimidinyl or pyrazinyl;
R₄ represents hydrogen, lower alkyl or halogen; or
(b) Py denotes 5-pyrimidyl, R₅ denotes —N(R₁)—C(O)—R₂,
R₁ is hydrogen, R₂ is [[(3S)-3-(dimethylamino)-1-pyrrolidinyl]methyl]-3-(trifluoromethyl)phenyl and R₄ is methyl;
or a pharmaceutically acceptable salt of such a compound.

6. The method according to claim 5, wherein the pyrimidylaminobenzamide derivative is 4-methyl-3-[[4-(3-pyridinyl)-2-pyrimidinyl]amino]-N-[5-(4-methyl-1H-imidazol-1-yl)-3-(trifluoromethyl)phenyl]benzamide.

7. The method according to claim 5, wherein the pyrimidylaminobenzamide derivative is employed in the form of its hydrochloride monohydrate.

8. The method according to claim 5, wherein the disorders mediated by ZAK is selected from hemolytic uremic syndrome, cardiac hypertrophy, cardiac fibrosis progression and ovarian cancer.
